(19) United States
(12) Patent Application Publication
(10) Pub. No.: US 2022/0195540 A1
(43) Pub. Date: Jun. 23, 2022

(54) MULTIPLEX REAL-TIME RT-PCR METHOD FOR THE DIAGNOSIS OF SARS-COV-2 BY TARGETING VIRAL E, RDRP AND HUMAN RP GENES OR VIRAL N2, RDRP AND HUMAN RP GENES

(71) Applicant: Imam Abdulrahman Bin Faisal University, Dammam (SA)

(72) Inventors: Huseyin TOMBULOGLU, Dammam (SA); Ebtesam AL-SUHAIMI, Dammam (SA)

(73) Assignee: Imam Abdulrahman Bin Faisal University, Dammam (SA)

(21) Appl. No.: 17/230,315

(22) Filed: Apr. 14, 2021

Related U.S. Application Data
(60) Provisional application No. 63/129,903, filed on Dec. 23, 2020.

(51) Int. Cl. C12Q 1/70

(52) U.S. Cl. CPC C12Q 1/701; C12Q 2600/158

(57) ABSTRACT

A method for detecting SARS-CoV-2 RNA or cDNA in a sample comprising real-time reverse transcription polymerase chain reaction that specifically amplifies and detects nucleic acid sequences amplified by primers to human RP gene, and SARS-CoV-2 RdRP and E, or SARS-CoV-2 RdRP and N2 genes. Specific primers and fluorescent probes that amplify and detect specific segments of human RP gene and SARS-CoV-2 RdRP and E, or SARS-CoV-2 RdRP and N2 genes with high sensitivity and efficiency compared to conventional methods.

### CROSS-REFERENCE TO RELATED APPLICATIONS

This application claims priority to U.S. Provisional Application No. 63/129,903 filed Dec. 23, 2020 which is hereby incorporated by reference for all purposes.

### ACKNOWLEDGMENT

This work was funded by Institute for Research and Medical Consultations (IRMC) under the project number 2020-IRMC-S-3 and by institutional fund/Ministry of Education no #Covid19-2020-026-IRMC Priority of the Medical and Health Sciences/Clinical Medicine.

### REFERENCE TO A SEQUENCE LISTING

In accordance with 37 CFR § 1.52(e)(5), the present specification makes reference to a Sequence Listing (submitted electronically as a .txt file named “534612US_ST25.txt”. The .txt file was generated on Mar. 26, 2021 and is 3.21 kb in size. The entire contents of the Sequence Listing are herein incorporated by reference.

### BACKGROUND OF THE INVENTION

### Field of the Invention

The invention relates to the fields of virology and molecular biology, specifically to RT-PCR-based methods of detecting SARS-CoV-2 virus.

### Description of Related Art

The outbreak of novel Betacoronavirus, SARS-CoV-2, that began in Wuhan, China in December 2019, has spread rapidly to multiple countries as a global pandemic. As of Mar. 26, 2021, about 125 million people were confirmed with SARS-CoV-2 infection and 2.7 million had died. The increasing number of infections worldwide necessitates the need for a less-invasive, reliable, and fast diagnostic tool to facilitate testing for exposure and infection with SARS-CoV-2.

Various diagnostic kits have been investigated including multiplex RT-PCR, CRISPR/Cas12, CRISPR/Cas3, lateral flow immunoassay, paper-based biomolecular sensors, SHERLOCK one pot testing and DNA aptamer based systems. Each of these methods has its own strong and weak points in terms of sensitivity and specificity. Among these methods, nucleic acid amplification-based tests are the most common for the diagnosis of SARS-COV-2. The US Food and Drug Administration (FDA) has approved at least 196 molecular diagnostic tests for the detection of SARS-CoV-2 nucleic acids under Emergency Use Authorization (EUA) (hypertext transfer protocol secure://www.fda.gov/medical-devices/coronavirus-disease-2019-covid-19-emergency-use-authorizations-medical-devices/vitro-diagnostics-euas). However, many existing nucleic amplification tests lack sensitivity, specificity for SARS-CoV and its variants, or speed.

Taking into account the limitations of prior methods of detection, the inventors sought to develop and clinically test an efficient and accurate system of multiplex real-time reverse transcription polymerase chain reaction (rRT-PCR) for the detection of SARS-COV-2 that can detect SARS-CoV-2 and its variants with a shorter reaction time and less effort. As disclosed herein, the inventors developed and tested two parallel multiplex systems one involving detection of the viral RdRP and E genes and the other involving detection of RdRP and N2. Both systems employ a human gene (RP) as an internal control.

### BRIEF SUMMARY OF THE INVENTION

The invention pertains to a highly sensitive, efficient and convenient way to detect SARS-CoV-2 RNA using reverse-transcription, real-time polymerase chain reaction to detect specific segments of the human RP gene (as a control) and SARS-CoV-2 RdRP and E genes, or SARS-CoV-2 RdRP and N2 genes using specially designed primers and probes.

Aspects of the invention include but are not limited to the following:

A multiplex real-time reverse-transcription polymerase chain reaction (rRT-PCR) method for detecting SARS-CoV-2 virus in a sample comprising, consisting essentially of, or consisting of: contacting cDNA produced from SARS-CoV-2 RNA with primers that amplify human RP, viral RdRP, and viral E or N2 genes, dNTPs, and a DNA polymerase under conditions suitable for amplification of the cDNA; contacting the amplified cDNA with fluorescent detection probes that bind to amplified human RP, viral RdRP, and viral E or N2 genes; and measuring gene-specific fluorescence which indicates the presence of SARS-CoV-2 RNA in the sample. A pseudoviral synthetic RNA is used as a positive control RNA. This method may conveniently be performed in a real-time thermocycler.

The control value may be taken from the positive control in a separate reaction tube. For example, a pseudoviral RNA including RdRP, N2 and E genes may be used as a positive control. In some embodiments, SARS-CoV2 RNA isolated from a COVID-19 positive individual can be used as a positive control.

Diagnosis of SARS-CoV-2 is preferably based on the fluorescent signal obtained before the 37^{th} PCR cycle, which shows the amplification of target viral genes and hence the presence of the virus.

In some embodiments of this method the cDNA is produced by isolating RNA from a sample, such an aspirate from the nose or respiratory system of a subject, from mucous, blood, plasma, or serum, or other biological samples from a subject, and reverse transcribing SARS-CoV-2 RNA (or control RNA). In a preferred embodiment, the RNA is obtained from a nasopharyngeal swab and/or a nasopharyngeal/oral swab. Samples may be stored or transported in a suitable medium, such as in Virus Liquid Transport Medium (VTM, Copan, USA), and preferably, kept refrigerated for not more than 8 hours.

In some embodiments, a commercial kit for reverse transcription may be used, such as, but not limited to, VitaScript™ FirstStrand cDNA Synthesis Kit which includes VitaScript™ Enzyme Mix and 5× VS Reaction Buffer, the buffer containing dNTPs.

It is unnecessary to use random hexamers or oligo-T primers. Preferably, gene-specific primers are used to reverse-transcribe SARS-CoV-2 and human RP-RNA as these offer the most specific priming for reverse transcription.

Reverse transcription to provide SARS-CoV-2 cDNA may be performed as a separate step or may be conducted simultaneously with other PCR steps, such as along with initial amplification of SARS-CoV-2 cDNA.

The method disclosed herein is preferably performed as a one-step RT-PCR method where reverse transcription of RNA and the amplification of genes by DNA polymerase occur simultaneously in the same reaction tube.

In one embodiment, SARS-CoV-2 cDNA (or control cDNA) is produced by reverse transcribing purified or isolated SARS-CoV-2 RNA or human RNA using an M-MLV reverse transcriptase, which is reactive at 42° C., which has RNAse H activity, but which has no detectable 3′ to 5′ exonuclease activity. One example of such a reverse transcriptase is the M-MLV reverse transcriptase available from Procomcure Biotech (VitaScript™ Reverse Transcriptase). Detailed instructions for use of a reverse transcriptase (and materials needed) include those available at hypertext transfer protocol secure://shop.procomcure.com/wp-content/uploads/2019/07/91-014-FirstStrand_cDNA_Synthesis_Kit_ISO.pdf (incorporated by reference, last accessed Mar. 26, 2021).

The DNA polymerase used may be a Taq DNA polymerase that has a fidelity of 1× Taq, which has a standard 1 min/kb reaction speed, exhibits a 3′-A product overhang, that has 5′ to 3′ exonuclease activity, that has undetectable 3′ to 5′ proofreading activity, and/or that has undetectable endonuclease activity. One example of such a polymerase is VitaTaq® DNA polymerase. Detailed instructions for use of DNA polymerase (and materials needed) include those available at hypertext transfer protocol secure://shop.procomcure.com/wp-content/uploads/2019/07/91-001-VitaTaq_2X_MM_ISO.pdf (incorporated by reference, last accessed Mar. 26, 2021). In some embodiments, the PCR reaction mixture comprises Triton-X 100 or dimethyl sulfoxide (DMSO), and Uracil-DNA glycosylase (UDG).

In a preferred embodiment, the methods disclosed herein employ primers that amplify human RP, viral RdRP, and viral E genes. These primers may comprise, consist essentially of, or consist of an RP forward primer AGATTTGGACCTGCGAGCG (SEQ ID NO: 1) and RP reverse primer GATAGCAACAACTGAATAGCCAAGGT (SEQ ID NO: 2); RdRP forward primer GTCATGTGTGGCGGTTCACT (SEQ ID NO: 4) and RdRP reverse primer CAACACTATTAGCATAAGCAGTTGT (SEQ ID NO: 5); or RdRP forward primer CCTCACTTGTTCTTGCTCGC (SEQ ID NO: 7) and reverse primer GCCGTGACAGCTTGACAAAT (SEQ ID NO: 8); and E forward primer GGAAGAGACAGGTACGTTAATA (SEQ ID NO: 10) and E reverse primer AGCAGTACGCACACAATCGAA (SEQ ID NO: 11). In some embodiments, these primers may be modified by deletion, insertion or substitution of 1, 2, 3 or 4 nucleotides. In other embodiments, one or more nucleotides may be modified to improve stability or other pharmacokinetic properties. These include substitution of 2′-O-methyl nucleotides, 2′-fluoro-nucleotides, 2′-amino nucleotides, and arabinose nucleotides for one or more nucleotides in the primer sequences described above or elsewhere herein.

In another preferred embodiment, the methods disclosed herein employ primers that amplify human RP, viral RdRP, and viral N2 genes. These primers may comprise, consist essentially of, or consist of an RP forward primer AGATTTGGACCTGCGAGCG (SEQ ID NO: 1) and RP reverse primer GATAGCAACAACTGAATAGCCAAGGT (SEQ ID NO: 2); RdRP forward primer GTCATGTGTGGCGGTTCACT (SEQ ID NO: 4) and RdRP reverse primer CAACACTATTAGCATAAGCAGTTGT (SEQ ID NO: 5); or RdRP forward primer CCTCACTTGTTCTTGCTCGC (SEQ ID NO: 7) and reverse primer GCCGTGACAGCTTGACAAAT (SEQ ID NO: 8); and N2 forward primer TGAAACTCAAGCCTTACCGC (SEQ ID NO: 13) and N2 reverse primer TATAGCCCATCTGCCTTGTG (SEQ ID NO: 14). In some embodiments, these primers may be modified by deletion, insertion or substitution of 1, 2, 3 or 4 nucleotides. In other embodiments, the nucleotides may be modified to improve stability or other pharmacokinetic properties. These include substitution of 2′-O-methyl nucleotides, 2′-fluoro-nucleotides, 2′-amino nucleotides, and arabinose nucleotides for one or more nucleotides in the primer sequences described above or elsewhere herein.

In some embodiments, the fluorescent detection probes are each labeled with a different fluorescent moiety and comprise, consist essentially of, or consist of: for RP: TTCTGACCTGAAGGCTCTGCGCG (SEQ ID NO: 3); for RdRP: CAGGTGGAACCTCATCAGGAGATGC (SEQ ID NO: 6) or GTGAAATGGTCATGTGTGGC (SEQ ID NO: 9); and for E: ACACTAGCCATCCTTACTGCGCTTCG (SEQ ID NO: 12).

Preferably, the fluorescent detection probes are each labeled with a different fluorescent moiety and consist of: for RP: ROX-TTCTGACCTGAAGGCTCTGCGCG-BHQ2 (SEQ ID NO: 3); for RdRP: FAM-CAGGTGGAACCTCATCAGGAGATGC-BHQ1 (SEQ ID NO: 6) or FAM-GTGAAATGGTCATGTGTGGC-BHQ1 (SEQ ID NO: 9); and for E: HEX-ACACTAGCCATCCTTACTGCGCTTCG-BHQ1 (SEQ ID NO: 12).

In another embodiment, the fluorescent detection probes are each labeled with a different fluorescent moiety and comprise, consist essentially of, or consist of: for RP: TTCTGACCTGAAGGCTCTGCGCG (SEQ ID NO: 3); for RdRP: CAGGTGGAACCTCATCAGGAGATGC (SEQ ID NO: 6) or GTGAAATGGTCATGTGTGGC (SEQ ID NO: 9); and for N2: ATCCATGAGCAGTGCTGAC (SEQ ID NO: 15).

Preferably, the fluorescent detection probes are each labeled with a different fluorescent moiety and consist of: for RP: ROX-TTCTGACCTGAAGGCTCTGCGCG-BHQ2 (SEQ ID NO: 3); for RdRP: FAM-CAGGTGGAACCTCATCAGGAGATGC-BHQ1 (SEQ ID NO: 6) or FAM-GTGAAATGGTCATGTGTGGC-BHQ1 (SEQ ID NO: 9); and for N2: HEX-ATCCATGAGCAGTGCTGAC-BHQ1 (SEQ ID NO: 15).

In preferred embodiments of the method disclosed herein will have a running time of 30, 35, 40, 45, 50, 55 or 60 minutes or less.

In other preferred embodiments, the method disclosed herein has a limit of detection (LOD) for the RdRP gene, E gene or N2 gene of SARS-CoV-2 of ≤1.25, 1.5, 2, 3, 4, 5, 6, 7, 8, 9, or 10 copy/μL.

In other preferred embodiments, the methods disclosed herein have an R² of at least 0.96, 0.97, 0.98, 0.99 for the RdRP, E or N2 genes of SARS-CoV-2 and/or an efficiency (E) of at least 0.96, 0.98, 0.98, or 0.99 for each of the RdRP, E and N2 genes.

Another aspect of the invention is a kit comprising reverse transcriptase, DNA polymerase, dNTPs a medium suitable for reverse transcription of SARS-CoV-2 RNA into cDNA, a medium suitable for amplification of cDNA, primers suitable for amplification of human RP and SARS-CoV-2 viral RdRP, and viral E genes, wherein said primers comprise, consist essentially of, or consist of RP forward primer AGATTTGGACCTGCGAGCG (SEQ ID NO: 1) and RP reverse primer GATAGCAACAACTGAATAGCCAAGGT (SEQ ID NO: 2); RdRP forward primer GTCATGTGTGGCGGTTCACT (SEQ ID NO: 4) and RdRP reverse primer CAACACTATTAGCATAAGCAGTTGT (SEQ ID NO: 5); or RdRP forward primer CCTCACTTGTTCTTGCTCGC (SEQ ID NO: 7) and reverse primer GCCGTGACAGCTTGACAAAT (SEQ ID NO: 8); and E forward primer GGAAGAGACAGGTACGTTAATA (SEQ ID NO: 10) and E reverse primer AGCAGTACGCACACAATCGAA (SEQ ID NO: 11); at least one container, and, optionally, a thermocycler and/or a fluorescence detector; and/or

primers suitable for amplification of human RP and SARS-CoV-2 viral RdRP, and viral E genes, wherein said primers comprise, consist essentially of, or consist of: RP forward primer AGATTTGGACCTGCGAGCG (SEQ ID NO: 1) and RP reverse primer GATAGCAACAACTGAATAGCCAAGGT (SEQ ID NO: 2); RdRP forward primer GTCATGTGTGGCGGTTCACT (SEQ ID NO: 4) and RdRP reverse primer CAACACTATTAGCATAAGCAGTTGT (SEQ ID NO: 5); or RdRP forward primer CCTCACTTGTTCTTGCTCGC (SEQ ID NO: 7) and reverse primer GCCGTGACAGCTTGACAAAT (SEQ ID NO: 8); and N2 forward primer GAAACTCAAGCCTTACCGC (SEQ ID NO: 13) and N2 reverse primer TATAGCCCATCTGCCTTGTG (SEQ ID NO: 14); and, optionally, at least one container, a thermocycler, a fluorescence detector, and/or instructions for use in detecting SARS-CoV-2. As described above, these primers may be modified by insertion, deletion or substitution of 1, 2, 3, or nucleotides or by chemical modification. In some embodiments, a kit comprises a real-time PCR system such as Applied Biosystems™, 7500 Fast Real-Time PCR system or other similar commercially available system. Preferably, all reagents including reverse transcriptase, DNA polymerase, dNTPs, and primers are combined as a single reaction medium. These reagents are thus ready-to-use and there is no need to combine them from separate reagent tubes.

In preferred embodiments of the method disclosed herein, the fluorescent detection probes are each labeled with a different fluorescent moiety and comprise: for RP: TTCTGACCTGAAGGCTCTGCGCG (SEQ ID NO: 3); for RdRP: CAGGTGGAACCTCATCAGGAGATGC (SEQ ID NO: 6) or TTCTGACCTGAAGGCTCTGCGCG (SEQ ID NO: 9); and for E: ACACTAGCCATCCTTACTGCGCTTCG (SEQ ID NO: 12); and/or fluorescent detection probes which are each labeled with a different fluorescent moiety and which comprise, consist essentially of, or consist of: for RP: TTCTGACCTGAAGGCTCTGCGCG (SEQ ID NO: 3); for RdRP: CAGGTGGAACCTCATCAGGAGATGC (SEQ ID NO: 6) or GTGAAATGGTCATGTGTGGC (SEQ ID NO: 9); and for N2: ATCCATGAGCAGTGCTGAC (SEQ ID NO: 15).

Another aspect of this technology, is directed to a method for preventing or treating an infection by SARS-CoV-2 comprising selecting a subject in need of vaccination or treatment for SARS-CoV-2 by detecting SARS-CoV-2 RNA in a biological sample from the subject according to the rRT-PCR methods disclosed herein, and vaccinating or treating the subject for SARS-CoV-2 when SARS-CoV-2 RNA is detected or vaccinating or prophylactically treating the subject when SARS-CoV-2 RNA is not detected. Vaccines include the Moderna, Pfizer-BioNTech, Johnson & Johnson, Astra-Zenica, Sputnik 5, and Sinopharm vaccines, as well as others approved for medical use. Pharmacological and biological treatments include administration of drugs such as remdesivir and other compounds having demonstrated anti-viral activity against coronaviruses or SARS-CoV, including in vitro or in vivo activity, anti-SARS-CoV-2 monoclonal or polyclonal antibodies, administration of oxygen, or use of a respirator.

Prevention includes both prophylaxis by vaccinate, passive immunization (e.g. anti-SARS-CoV-2 monoclonal or polyclonal antibody infusion), or pharmacological treatment, as well as isolation, use of masks, work from home, rest, hydration, over-the-counter medicines such as acetaminophen.

The foregoing paragraphs have been provided by way of general introduction, and are not intended to limit the scope of the following claims. The described embodiments, together with further advantages, will be best understood by reference to the following detailed description taken in conjunction with the accompanying drawings.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings below.

FIG. 1A. Cycle threshold (Ct) value of rRT-PCR repeats. Simplex assay targeting RdRP, E, and RP genes in separate reaction tubes.

FIG. 1B. Cycle threshold (Ct) value of rRT-PCR repeats. Multiplex or duplex assay amplifies two genes simultaneously: RdRP and RP.

FIG. 1C. Cycle threshold (Ct) value of rRT-PCR repeats. Multiplex or duplex assay amplifies two genes simultaneously: E and RP.

FIG. 1D. Cycle threshold (Ct) value of rRT-PCR repeats. Multiplex or triplex assay that targets two viral (RdRP and E) and one human internal control (RP) gene simultaneously.

FIG. 2A. The limit-of-detection (LOD) of RdRP gene, amplification plot. A serial dilution of synthetic RNA (10⁵, 10⁴, 10³, 10² and 10¹ copies/μL) was prepared.

FIG. 2B. The limit-of-detection (LOD) of RdRP gene, efficiency.

FIG. 2C. The limit-of-detection (LOD) of E gene, amplification plot. A serial dilution of synthetic RNA (10⁵, 10⁴, 10³, 10² and 10¹ copies/μL) was prepared.

FIG. 2D. The limit-of-detection (LOD) of E gene, efficiency.

FIG. 3A. Comparison of the E gene cycle threshold (Ct) value of COVID-19 positive samples using Cepheid's and the current (COV2-kit) assays.

FIG. 3B. The data show the Ct value of the E gene, which is the common gene in both assays. Comparison of Cepheid's N2 and COV2-kit's RdRP genes.

FIG. 3C. Comparison of Ct values of the target genes for Cepheid's E and N2. The red/dashed line shows a threshold value of 37, which is accepted as the upper limit for SARS-CoV-2 detection by CDC.

FIG. 3D. Comparison of Ct values of the target genes for COV2-kit's E and RdRP. The red/dashed line shows a threshold value of 37, which is accepted as the upper limit for SARS-CoV-2 detection by CDC.

FIG. 4A. Amplification curves of clinical samples detecting RP, RdRP and N2—SARS-CoV-2 ‘positive’ specimens.

FIG. 4B. Amplification curves of clinical samples detecting RP, RdRP and N2—SARS-CoV-2 ‘negative’ specimens.

FIG. 4C. The amplification curve of positive control.

FIG. 4D. The amplification curve of d negative control.

FIG. 5A. Standard curve for multiplex qRT-PCR analysis of RdRP primers. The template RNA was serial diluted with a range of 10⁵ to 10¹. Amplification plot.

FIG. 5B. Standard curve for multiplex qRT-PCR analysis of RdRP primers. The template RNA was serial diluted with a range of 10⁵ to 10¹. Efficiency.

FIG. 5C. Standard curve for multiplex qRT-PCR analysis of N2 primers. The template RNA was serial diluted with a range of 10⁵ to 10¹. Amplification plot.

FIG. 5D. Standard curve for multiplex qRT-PCR analysis of N2 primers. The template RNA was serial diluted with a range of 10⁵ to 10¹. Efficiency.

FIG. 6. The cycle threshold (Ct) scores of the same clinical samples tested either the current COV-2 assay or commercial kits. Each bar represents different genes which are RdRP and N2 for COV-2 assay; and N2 or S and RdRP or E for commercial kits.

FIG. 7A. Determination of the limit of detection (LOD) for RdRP primer. The 5×10⁴ copy/μl pseudoviral RNA was serially diluted. Amplification plot. The R² value of the trendline and the efficiency (E) of the standard curve were displayed on each graph. The error bars represent the standard deviation between the replicates.

FIG. 7B. Efficiency. RdRP primer.

FIG. 7C. Determination of the limit of detection (LOD) for N2 primer. The 5×10⁴ copy/μ1 pseudoviral RNA was serially diluted. Amplification plot. The R² value of the trendline and the efficiency (E) of the standard curve were displayed on each graph. The error bars represent the standard deviation between the replicates.

FIG. 7D. Efficiency. N2 primer.

FIG. 8. Schema. Genome structure of SARS-CoV-2 and the targeted genes to be used in multiplex rRT-PCR. Here, the RdRP and N2 genes are targeted. In another embodiment, the E gene would be targeted instead of the N2 gene.

### DETAILED DESCRIPTION OF THE INVENTION

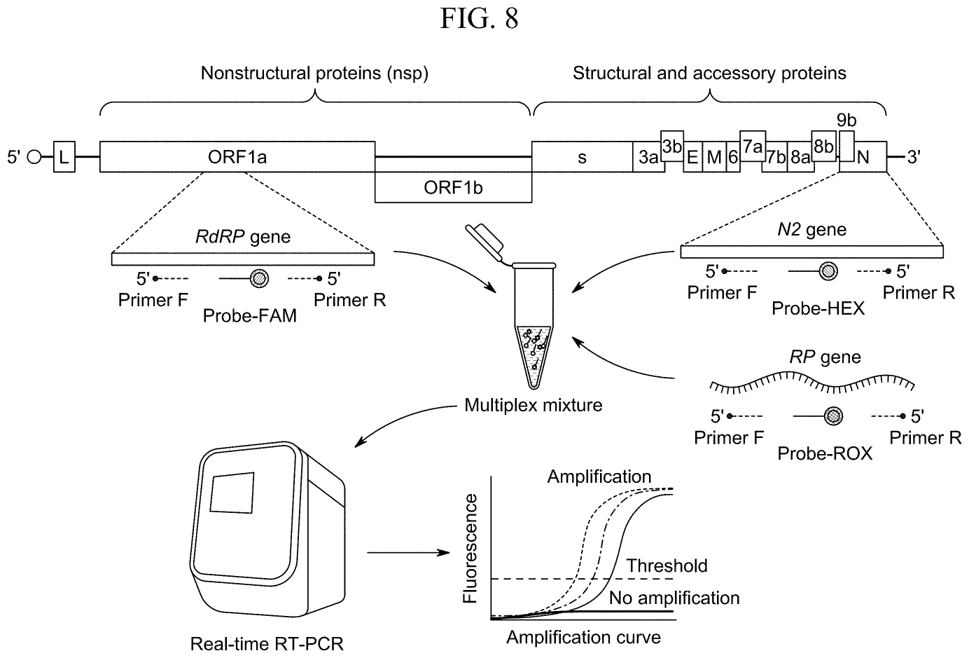

The invention pertains to a method for detecting SARS-CoV-2 RNA or cDNA in a sample comprising multiplex real-time reverse-transcription polymerase chain reaction that specifically amplifies and detects nucleic acid sequences amplified by primers to human RP gene, and SARS-CoV-2 RdRP and E, or SARS-CoV-2 RdRP and N2 genes. Specific primers and fluorescent probes that amplify and detect specific segments of human RP gene and SARS-CoV-2 RdRP and E, or SARS-CoV-2 RdRP and N2 genes with high sensitivity and efficiency compared to conventional methods. A general schema is shown in FIG. 8.

SARS-CoV-2 is a positive-sense single-stranded RNA ((+) ssRNA) virus. Its genome consists of 29,900 nucleotides (nt) enclosing five open reading frames (ORFs) (5′-3′); ORF lab polyprotein (P, 7,096 amino acids), spike glycoprotein (S, 1,273 amino acids), nucleocapsid protein (N, 419 amino acids), envelope protein (E, 75 amino acids), and membrane protein (M, 222 amino acids); see Liu et al., Promising methods for detection of novel coronavirus SARS-CoV-2, VIEW, 2020, 1, e4). Molecular phylogeny of SARS-CoV-2 has revealed two main macro-haplogroups, A and B, with more than 160 sub-branches representing virus strains of variable geographical origins worldwide and there are 483 unique variations among SARS-CoV-2 genomes with 40 variations only in the S glycoprotein and 6 non-synonymous mutations exist in at the receptor-binding domain (RBD).

The specific design of the primer/probe sequences targets the consensus regions of the selected genes. This allows them to recognize more SARS-CoV-2 variants. In some embodiments a consensus sequence may be evaluated to determine whether it recognizes a particular SARS-CoV-2 variant or may be modified to take into account gene sequences of particular SARS-Cov-2 variants.

rRT-PCR detecting human RP and viral RdRP and E genes. The outbreak of the new human coronavirus SARS-CoV-2 (also known as 2019-nCoV) continues to increase globally. Fast, reliable, and practical techniques are urgently needed to diagnose SARS-CoV-2 infection. The real-time reverse transcription polymerase chain reaction (rRT-PCR) is the most used technique in virus detection. However, possible false-negative and false-positive results produce misleading consequences in terms of the patient's condition. Also, the amplification of single gene targets reduces the reliability of this method for SARS-COV-2 detection. Accordingly, the inventors developed a multiplex rRT-PCR diagnostic method, which targets two viral genes (RdRP and E), or in an alternate embodiment (RdRP and N2), and one human gene (RP) simultaneously.

As disclosed herein, the inventors sought to develop and assess the performance of an efficient multiplex real-time reverse transcription polymerase chain reaction (rRT-PCR) for the detection of SARS-COV-2. The assay simultaneously targets two viral genes (RdRP and E) or RdRP and N2) and a human gene (RP) as internal control by using the Applied Biosystems 7500 Fast Real-Time PCR instrument (ABI, Thermo Fisher Sci). In addition, the clinical performance of the assay was evaluated on SARS-CoV-2 samples collected from COVID-19 positive patients and compared by using the GeneXpert Dx instrument (Cepheid, Sunnyvale, Calif., USA).

The reaction was tested by using pseudoviral RNA and human target mRNA sequences as template and the protocol was validated by using 14 clinical SARS-CoV-2 positive samples.

The results were in good agreement with the CDC authorized Cepheid's Xpert® Xpress SARS-CoV-2 diagnostic system (100%).

Unlike single gene targeting strategies, the current method provides the amplification of two viral regions at the same time of PCR reaction and it is unnecessary to repeat the assay for each gene. As a result, an accurate SARS-CoV-2 diagnostic assay was provided, which allows testing of 91 samples in 96-well plates in per run. The inventors consider that by targeting two viral genes and one human gene in the same rRT-PCR reaction the reliability and accuracy of rRT-PCR was increased with less run time and lower amounts of PCR reagents. This strategy provides a fast, reliable, and easy-to-use rRT-PCR method to diagnose SARS-CoV-2.

In one example, a traditional two-step or one-step simplex rRT-PCR reaction requires dNTPs in concentrations of about 10 mM each, about 2 U/μL of Taq polymerase, 100-200 U/μL of M-MLV reverse transcriptase enzymes, and buffers/reagents for each target gene amplification performed in a separate tube. In the method disclosed herein, the same of lesser amounts of enzymes and reagents (e.g., 5, 10, 20, 30, 40, 50 or >50% (in wt/v % or U/v %) are used providing for a more economical and cost-effective assay.

Polymerase chain reaction (PCR). PCR is used to amplify selected sections of DNA or RNA, such as sections representative of a particular SARS-CoV-2 or human gene. Typically, a nucleic acid, such as DNA or cDNA is denatured, for example, by heating to 94° C. to form single strands. Two primers are added to the denatured strands at a lower annealing temperature, such as about 54° C. to permit binding of the primers to the single strands. The sequences of the two primers correspond to the beginning and ending of a target sequence on the single strand. The primer sequences are extended by a heat-stable polymerase in the presence of dNTPs, typically at a temperature of about 72° C., thus forming a double-stranded nucleic acid. During a single cycle as described above, a single segment of double-stranded DNA is amplified into two pieces of double-stranded DNA. These two pieces are then available for a next cycle of amplification; the number of copies of the target sequences is exponentially increased during subsequent cycles. The entire cycling process of PCR has been automated and can be completed in just a few hours. It is directed by a machine called a thermocycler, which is programmed to alter the temperature of the reaction every few minutes to allow DNA denaturing and synthesis.

Reverse-transcription-PCR is similar to PCR and includes an initial step of synthesizing cDNA from RNA by reverse transcription of a target RNA. The cDNA produced is then amplified using PCR.

Real-time polymerase chain reaction (real-time PCR), also known as quantitative Polymerase Chain Reaction (qPCR), is a laboratory technique of molecular biology based on the polymerase chain reaction (PCR). It monitors the amplification of a targeted DNA molecule during the PCR (i.e., in real time), not at its end, as in conventional PCR. Real-time PCR can be used quantitatively (quantitative real-time PCR) and semi-quantitatively (i.e., above/below a certain amount of DNA molecules) (semi-quantitative real-time PCR).

Real-time reverse-transcription PCR (rRT-PCR) combines the features of Reverse transcription PCR and Real-time PCR. Examples of rRT-PCR using viral RNA and control mRNA are disclosed below.

Reverse transcription of viral or control RNA. A preferred reverse transcription is contained in VitaScript® Enzyme mix with M-MLV reverse transcriptase, which is commercially available and incorporated by reference to hypertext transfer protocol secure://shop.procomcure.com/wp-content/uploads/2019/07/91-014-FirstStrand_cDNA_Synthesis_Kit_ISO.pdf which describes reverse transcriptase, reagents and methods for making cDNA from viral RNA. In some embodiments, Taq DNA polymerase may be used for the amplification of viral RNA; see Bhadra, S. et al., BIOCHEMISTRY, 2020, 59)49), 4638-4645 (incorporated by reference).

DNA polymerases suitable for use in PCR are commercially available and include Taq (Thermus aquaticus) polymerase as well as other heat-stable polymerases. One preferred DNA polymerase is VitaTaq® HS polymerase which is used in the examples which follow. VitaTaq® HS DNA Polymerase is a thermostable DNA Polymerase that synthesizes DNA from single stranded templates. The enzyme has a 5′-3′ polymerase activity and low 5′-3′ exonuclease activity. Additional description of this heat-stable polymerase and methods and reagents, such as dNTP mix and 10×PCR buffer, suitable for performing PCR using it are incorporated by reference to hypertext transfer protocol secure://shop.procomcure.com/wp-content/uploads/2019/07/91-125-VitaTaq-HS-PCR-Kit_ISO.pdf (last accessed Mar. 25, 2021.

Dyes/Quenchers. One skilled in the art can select appropriate reporter and detector dyes for use in rRT-PCR. Typically, dyes are selected that are compatible with the detector instrument. Which must be capable of detecting the emission spectrum for each dye being used, e.g., dyes for each of the amplicons being detected, and quencher dyes; see hypertext protocol secure://www.idtdna.com/pages/education/decoded/article/qpcr-probes-selecting-the-best-reporter-dye-and-quencher (incorporated by reference, last accessed Mar. 25, 2021). Such reporter dyes include FAM, TET, HEX, JOE, Cy3, TAMRA, ROX, LC Red 610, Texas Red, LC 640 and Cy5. In a preferred embodiment, FAM, VIC or HEX, and ROX labelled probes are used to identify RP, RdRP, and E or N2 amplicons. Preferably, the quencher is a broadly adsorbing dark non-fluorescent quencher which permits use of multiple reporter dyes with the same quencher. Quenchers include so-called Black hole quenchers, ZEN quencher, Iowa Black FQ and RQ. The probes described herein preferably use quencher BHQ1. Dyes and quenchers described herein include all those commercially available on the filing date of this application.

Ct value. The cycle threshold (Ct) value of a reaction is defined as the cycle number when the fluorescence of a PCR product can be detected above the background signal. In order to calculate the Ct value, it is necessary to draw a horizontal line (threshold) on the amplification plot. The Ct value is associated with the amount of PCR product in the reaction. The lower the Ct value, the more PCR product that is present. This is because it takes fewer PCR cycles for that product to be detected over the background signal. In a preferred embodiment, the method as disclosed herein amplifies SARS-CoV-2 RNA/cDNA with a Ct (cycle threshold) value ranging from at least 10, 15, 20, 25, 30, 31, 32, 33, 34, 35, 36, 37, or <38.

To detect positivity as disclosed herein, a Ct range between 10 and 38 is used. A Ct value greater than 38 means the sample is negative for SARS-CoV-2. In conventional PCRs, a sample having a Ct value of <40. WHO-advised protocol recommends that a gene having a Ct value≤37 is accepted as positive, a Ct value>40 is accepted as negative and a gene having a value 35<Ct<37 is considered to be in diagnostically gray zone.

R² value. This coefficient only takes values between 0 and 1. R² is used to assess the fit of the standard curve to the data points plotted. The closer the value to 1, the better the fit. An R² value>0.99 provides good confidence in correlating two values.

Efficiency. Quantitative polymerase chain reaction (or qPCR) is a well-established assay for nucleic acid quantification and is still regarded as the method of choice in most areas of molecular biology. Though different types of qPCR quantification exist (absolute and relative), determining the amplification efficiency should be among the first things to do when setting up a qPCR assay. Understanding efficiency and how to calculate it is crucial for accurate data interpretation. Ideally, the number of molecules of the target sequence should double during each replication cycle, corresponding to a 100% amplification efficiency. Similarly, if the number of replicated molecules is less than double this is due to poor efficiency—below 100%. The most common reasons for lower efficiencies are bad primer design and non-optimal reagent concentrations or reaction conditions. Secondary structures like dimers and hairpins or inappropriate melting temperatures (Tm) can affect primer template annealing which results in poor amplification. Since each additional dilution contains appropriately lower starting amounts of DNA, differences occur between Ct values in serially diluted samples (see below). In preferred embodiments, the method disclosed herein has an efficiency of at 0.99 or 1.00. The PCR efficiency of both PCR assays disclosed herein are higher than 0.999 and in the range between 0.999 and 1.002. These values show coherence and harmony of the reagents used in the assay reactions. It is rare to attain this compatibility in multiplex PCR reactions with high efficiency and without formation of secondary structures. In general, a PCR efficiency score higher than 0.95 is acceptable, and as noted above, in the assays disclosed herein is very close to 1 indicating a great PCR efficiency.

### Example 1

### Detection of RP, RdRP and E Genes

Alignment of SARS-nCoV-19 genome sequences. Genomic sequences of all SARS-nCoV-19 types that have been sequenced worldwide were downloaded from the database of GISAID (Global Initiative on Sharing All Influenza Data, hypertext transfer protocol secure://wordldwide web.gisaid.org (incorporated by reference, last accessed Mar. 25, 2021). The comparative analyses by aligning the sequences at base level were made with bioinformatics programs such as “Blast”, “Muscle”, and “ClustalW2”. More than 100 annotated genomes, whose genome sequence information have been determined were selected. These genomes included those in samples from Europe, America, and Asia. In this way, virus gene targets adjusted as sensitive, specific, and accurate as possible. The accession date for the accessed sequences was May 5, 2020.

The full genome sequences of 100 SARS-CoV-2 viruses originated in different regions including but not limited China (hCoV-19/Wuhan/IVDC-HB-04/2020, hCoV-19/Wuhan/Hu-1/2019), Thailand (hCoV-19/Thailand/61/2020), USA (hCoV-19/USA/AZ1/2020), England (hCoV-19/England/01/2020), Belgium (hCoV-19/Belgium/GHB-03021/2020), France (hCoV-19/France/IDF-0515-isl/2020), Germany (hCoV-19/Germany/BW-ChVir-1577/2020), Thailand (hCoV-19/Canada/BC_37_0-2/2020) etc, were downloaded from the GISAID database. Care was taken to select virus varieties from different regions as possible. The primer and probe sequences were determined from the conserved regions of those aligned genomes. In the selection of SARS-COV-2 primers, attention was paid to the selection of genome regions that differ from other SARS-COV viruses. Therefore, primers are specific to this virus only, and exempt from possible cross reactions with other virus strains.

The method as disclosed herein does not target on gene only, but three genes simultaneously. This increases the probability of including any future mutations in these genes, especially in the N gene, which would not be identified if only a single gene were used The method doesn't target one gene only, but three genes as described above to give great chance to include any future mutations in these genes which will not be identified if we used one gene only.

Multiplex primer/probe design. Multiplex PCR compatible primer and probe arrays specific to viral and human gene targets were designed using programs such as “Primer Pooler”, “PrimerPlex”, and “Primer3”. The sequences were aligned by using the online MUSCLE program (hypertext transfer protocol secure://www.ebi.ac.uk/Tools/msa/muscle/) with the default setting (incorporated by reference, last accessed Apr. 5, 2021). The consensus sequences (%100 alignments) corresponding to the target genes were selected for primer and probe design. To find and validate the best primer/probe sequence, the programs “Primer Pooler”, “PrimerPlex”, and “Primer3’ were used.

Fluorescein amidites (FAM) labeled probe for the viral RdRP gene, a hexachloro-fluorescein (HEX) labeled probe for the viral E gene, and a carboxyrhodamine (ROX) stained probe for the human RP gene were designed and synthesized. BHQ1 describes a quencher dye. In some embodiments, VIC may be used interchangeably with HEX.

The concentration of each primer or probe is stated below.

| | |
|---|---|
| Probes: 5′-dye-target sequence-dye-3′ | Concentration: 5 μM |
| Primers: Forward: 5′-target sequence-3′ | Concentration: 20 μM |
| Reverse: 5′-target sequence-3′ | Concentration: 20 μM |

TABLE 1
The sequence of primer and probe
sets used in the PCR.

| | | SEQ |
|---|---|---|
| Primer/ | Sequence | ID |
| probe | (5′-3′) | NO: |
| RdRP-F | GTCATGTGTGGCGGTTCACT | 4 |
| RdRP-R | CAACACTATTAGCATAAGCAGTTGT | 5 |
| RdRp-P | FAM-CAGGTGGAACCTCATCAGGAGATGC- | 6 |
| | BHQ1 | |
| E-F | GGAAGAGACAGGTACGTTAATA | 10 |
| E-R | AGCAGTACGCACACAATCGAA | 11 |
| E-P | HEX-ACACTAGCCATCCTTACTGCGCTTCG- | 12 |
| | BHQ1 | |
| RF-F | AGATTTGGACCTGCGAGCG | 1 |
| RF-R | GATAGCAACAACTGAATAGCCAAGGT | 2 |
| RP-P | ROX-TTCTGACCTGAAGGCTCTGCGCG- | 3 |
| | BHQ2 | |

RNA isolation. In rRT-PCR, RNA extraction is a vital pre-analytical process. It is mainly carried out using a commercially available RNA extraction kit. An appropriate kit may be selected and acquired by one skilled in the art. RNA isolation kits include those available from QIAGEN, hypertext transfer protocol secure://www.qiagen.com/us/applications/molecular-biology-research/rna-resource-center?cmpid=PC_QF_NON_rna-purification-traffic_0321_SEA_GA (last accessed Mar. 29, 2021, incorporated by reference) or AGILENT, hypertext protocol secure://www.agilent.com/cs/promotions/misc/brochure-qPCR.pdf (last accessed Mar. 29, 2021, incorporated by reference).

Viral RNA was extracted from nasopharyngeal swabs in virus transport medium (VTM) which were sent to the microbiology laboratory at King Fahd Hospital of the University (KFHU), AL Khobar for SARS-CoV-2 detection. RNA extraction was performed from 280 of the VTM using the QIAamp Viral RNA Mini kit (Qiagen, Hilden, Germany) according to the manufacturer's instructions.

RT-qPCR reaction. The reaction mixture (20 μL) includes the following reagents: 2 of 10× Buffer, 0.25 μL of dNTPs (10 mM each), 0.2 μL of uracil-DNA glycosylase (UDG) (1 U/μL), 0.4 μL of VitaTaq® HS polymerase (2 U/μL), 0.05 μL VitaScript® Enzyme mix including M-MLV (Procomcure, Austria), 0.05 μL of Triton™ X-100 (molecular biology grade, Merck), the primer and probe mixture, and RNase/DNase-free ddH₂O up to 20 μL. The mixture for the primer and probe is varied according to the kit design. For the multiplex kit that simultaneously targeting the three genes, the final concentration of the primers/probes were adjusted as follow:

1) 10 pM for RdRP-F, 13 pM for RdRP-R, and 4 pM for RdRP-P

2) 4 pM for E-F, 4 pM for E-R, and 2 pM for E-P

3) 10 pM for RP-F, 3.75 pM for RP-R, and 4 pM for RP-P

The mixture was dispensed in 96-well plates (MicroAmp™ Fast Optical 96-well reaction Plate 0.1 mL, Applied Biosystems) and sealed with optical film (MicroAmp™ Optical Adhesive Film, Applied Biosystems). Pseudoviral RNAs including viral RdRP and E gene and human RNaseP (RP) mRNA sequences were used as the positive template. Meanwhile, RNase/DNase-free ddH₂O was added to the negative control tubes to check any contamination or primer dimer.

Quantitation experiments were performed in a real-time PCR instrument (Applied Biosystems™, 7500 Fast Real-Time PCR System). Before the operation, the instrument was calibrated by using Applied Biosystems™ 7500 Fast Real-Time PCR Systems Spectral Calibration Kit. Then, the qPCR reaction conditions were adjusted as follow: 1) Reverse transcription at 45° C. for 5 min, 2) Pre-denaturation at 95° C. for 30 sec, 3) 40 cycles of denaturation at 95° C. for 5 sec and amplification at 60° C. for 30 sec. The reporter dye channel sets as FAM for viral RdRP gene; and HEX for E gene; and ROX for human RNAseP (RP) gene. For the Applied Biosystems™ real-time PCR instrument (7500 and StepOne models), set to “passive reference” dye as “none”.

Amplification efficiency. To find out the amplification efficiency (E) of the genes, a standard curve from the dilution series of templates was prepared. Ct values versus the logarithmic amount of the template was plotted. The amplification efficiency was obtained by using the following equation:

E=100×(10^{-1/slope})

Validation of the assay by Xpert Xpress SARS-CoV-2. The same 14 nasopharyngeal swabs used for the validation of the current assay were also tested for SARS-CoV-2 using the Xpert Xpress SARS-CoV-2 kit (Cepheid, Sunnyvale, Calif., USA). About 300 μL of the VTM were transferred to the Xpert Xpress SARS-CoV-2 kit cartridge. The kit includes direct RNA extraction and rRT-PCR targeting the E and N2 gene fragments of the SARS-CoV-2. The assay was run on the GeneXpert Dx instrument (Cepheid, Sunnyvale, Calif., USA).

Data analysis. The results were evaluated by determining the amplification curve of the target gene and internal control gene. For the ABI 7500 device, the cycle threshold (Ct or Cq) line was automatically adjusted to ensure that the curves are all straight position. For this purpose, ABI 7500 software (v2.3) was used. The cycle threshold number≤38 with a sigmoidal curve is accepted as ‘positive’.

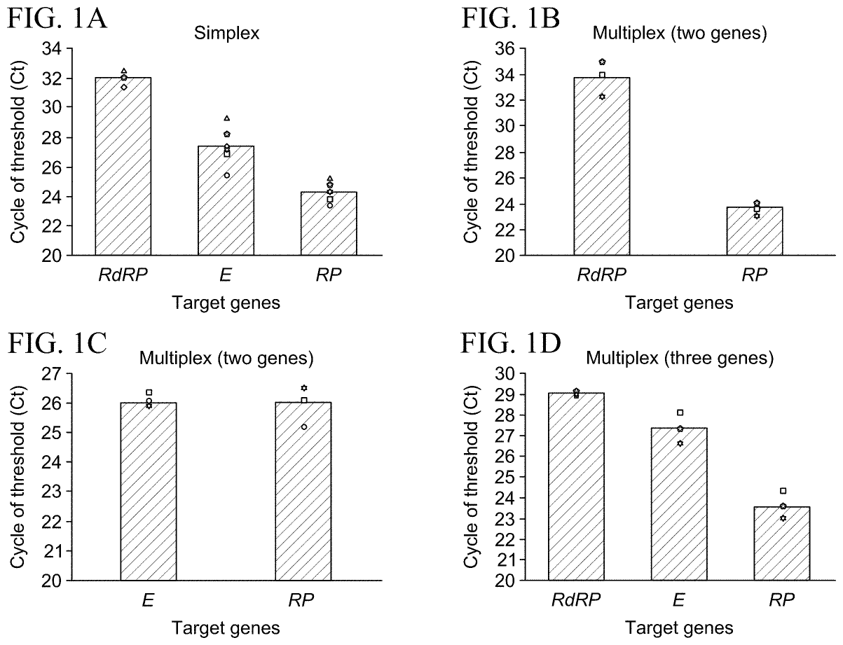

Simplex rRT-PCR standardization. Before multiplexing, all targeted gene primer/probe set, and RT-PCR reagents were tested in simplex rRT-PCR. The quantity of SARS-CoV-2 specific E and RdRP gene primer and probe set were optimized by using 10⁵ copy/μL of synthetic viral RNA as template. Before the reactions, the rRT-PCR instrument (Applied Biosystems™, 7500 Fast Real-Time PCR System) was calibrated to get the best fluorescent performance. The simplex reactions were repeated six times for two viral E and RdRP genes and one internal control (IC) gene RP. The cycle threshold (Ct) value with standard deviations (SD) were 24.1±1.05, 27.1±1.5, and 31.5±1.0 for RP, E and RdRP, respectively (FIG. 1A).

Duplex rRT-PCR standardization. The primer and probe sets were designed to detect one viral (E or RdRP) and one human IC gene (RP) at the same rRT-PCR reaction. Two different reaction mixtures were designed including the primer and probe sets either for RdRP with RP, or E with RP. Triplicate reactions yielded the Ct value as 23.6±0.77 and 32.3±1.8 for RP and RdRP genes, respectively (FIG. 1B). In the second reaction mixture, the Ct value was detected as 25.7±0.84 and 25.5±0.73 for RP and E genes, respectively (FIG. 1C). The Ct value of both reactions are less than 38, which is the recommended limit of CDC (Center of Disease Control and Prevention). FIG. 1D shows a multiplex or triplex assay that targets two viral (RdRP and E) and one human internal control (RP) gene simultaneously.

Multiplex (triplex) rRT-PCR standardization. Multiplex rRT-PCR protocol was set up for the amplification of three genes (E, RdRP, and RP). Three primer and probe sets for each gene were combined in the same reaction tube. Sigmoidal amplification curves were obtained with an average Ct value±SD as 28.8±0.51, 27.3±0.75, and 23.6±1.04 for RdRP, E, and RP genes, respectively. This shows that the assay is capable to detect three genes in the same reaction tube.

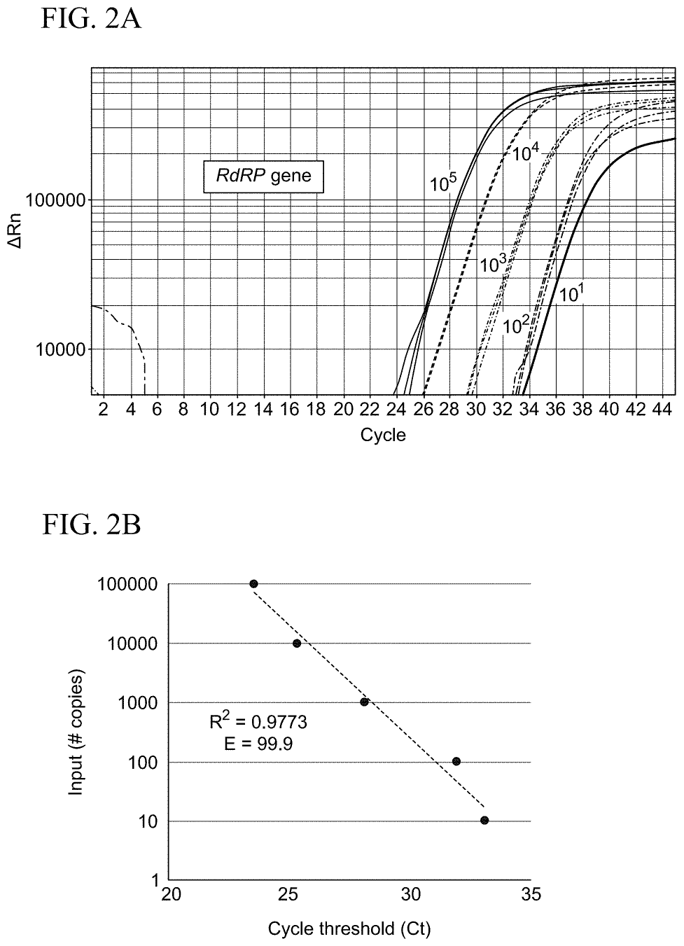

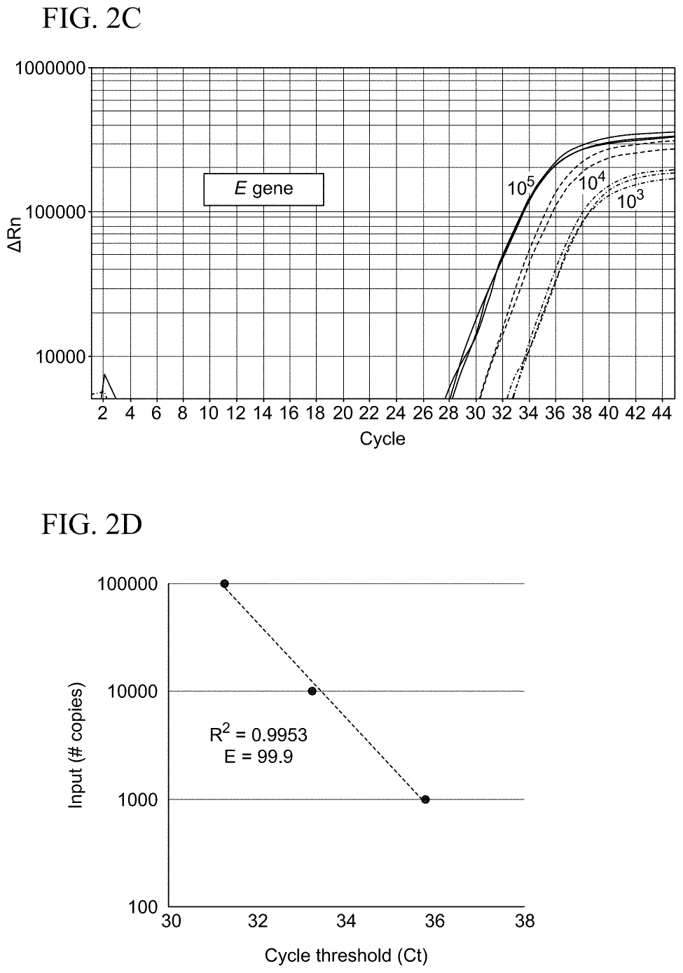

Limit-of-detection (LOD) and rRT-PCR efficiency. A serial dilution of synthetic RNA (10⁵, 10⁴, 10³, 10² and 10¹ copies/μL) was prepared to find the limit-of-detection (LOD) for RdRP and E genes. The amplification plots, the amplification efficiencies (E), and R² score are represented in FIGS. 2A-2D. The LOD of RdRP gene was ≥10 copy/μL. Nevertheless, the LOD of E gene was ≥10³ copy/μL. The E value of RdRP and E genes were determined as 99.9. The R² scores were determined as 0.977 for the RdRP and 0.995 for the E gene.

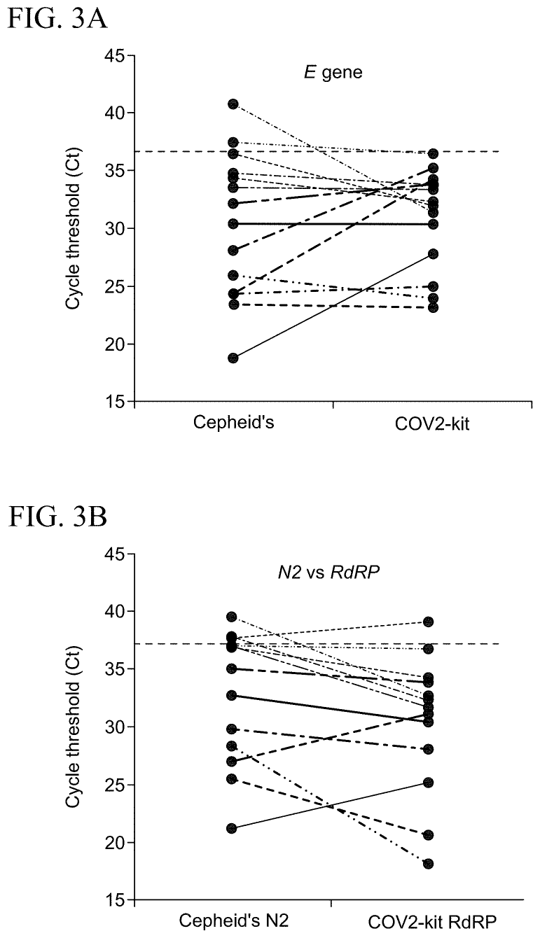

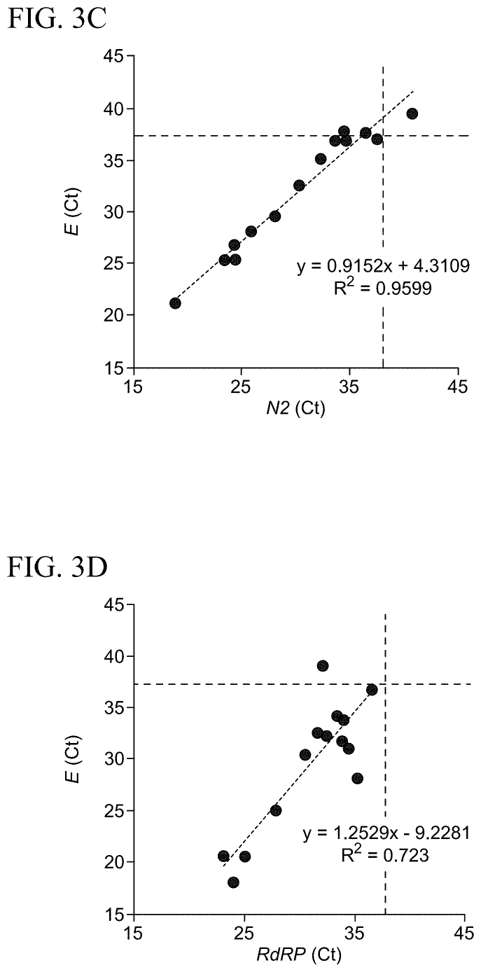

Validation of the assay using SARS-CoV-2-positive samples detected by Cepheid's system. To validate the outcome of multiplex rRT-PCR assay (named as COV2-kit) in the clinical SARS-CoV-2-positive samples, we compared our results by using Cepheid's GeneXpert® System. For this purpose, first the nasopharyngeal swabs were collected from COVID-19 infected patients between the 1^{st} and the 11^{th} of November 2020 at King Fahd Hospital of University (KFHU), AL Khobar. Then, the samples were kept in VTC medium and 300 μL of the solution were directly transferred to the Cepheid's GeneXpert® cartridge. The samples were run by using Xpert® Xpress SARS-CoV-2 detection kit. The SARS-CoV-2 positive samples were selected for viral RNA extraction (n=14) (QIAamp Viral RNA Mini Kit, Qiagen, Germany). Then, the extracted RNA was used as template to test our assay. The comparative Ct performances of each assay was shown in FIGS. 3A and 3B. The COV2-kit detected all confirmed SARS-CoV-2 positive samples. All Ct values of the E gene are below the threshold accepted by the CDC (≤37). (FIG. 3A). For the RdRP gene, only one clinical sample was out of the threshold, which is consistent with Cepheid's assay (FIG. 3B).

With the accumulation of many thousands of viral gene sequences, the improvement of the virus detection methods becomes high clinical demand. This study established a multiplex rRT-PCR assay simultaneously targeting two viral (RdRP and E) and one human (RP) genes in a single reaction tube. The assay was named COV2-kit. Thanks to the specific probes that were labelled with different fluorescence dyes (VIC, ROX, and FAM), the gene amplifications can be identified in the same reaction tube by using different filters of the Applied Biosystems™, 7500 Fast Real-Time PCR System. Three different approaches were performed in order to optimize the protocol: simplex (targets single gene), duplex (simultaneously targets two genes) and triplex (simultaneously targets three genes). For this purpose, a synthetic viral template with mRNA of RP gene was used. The Ct values of the reactions are ranged in between 24 to 34.

According to the CDC (Center of Disease Control and Prevention) recommendations, the acceptable Ct value should not exceed 37 to accept the sample as positive. The Ct values in all tested approaches (simplex, duplex or triplex) were under this threshold. The LOD (limit-of-detection) for RdRP and E genes were at least 10¹ and 10³ copy/μL, respectively. The primers and probe for the RdRP gene were found to be more sensitive than these for the E gene. The viral load of the COVID-19 patients is the critical factor for the test efficiency. It can be concluded that RdRP gene provides the maximum detection capability on the patients having low viral load (≥10¹ copy/μL). In addition, the patients with a viral load of ≥10³ copy/μL can be detected by using the E gene as target. Thus, the samples with a low viral load can be detected by using these two sensitive gene targeting approaches. In addition to these viral genes, the reaction includes a human gene target, RP, as an internal control (IC). In general, the IC gene is tested in a separate tube for each reaction which decreases the sample size to be tested and increase the expenses. By using current strategy, the number of reactions per sample is reduced. For instance, the assay allows testing 91 patients in 96-well plates per run, thus provides less time and save expensive RT-PCR reagents. Fast, reliable, high-sensitivity and low-cost SAR-COV-2 detection is achieved by designing and using effective primer/probe sets. Although the focus here is on SARS-CoV-2, this approach can also be used to detect other types of viruses.

The COV2-kit assay was tested to verify its performance on clinical samples (n=10). In addition, the results were compared by using Cepheid's GeneXpert® System that uses Xpert® Xpress SARS-CoV-2 detection kit (FIG. 3). Rather than our strategy that targets the RdRP and E genes, the Xpert® Xpress SARS-CoV-2 detects N2 and E genes. The Ct value of the E gene was in between 18 and 41 using the Cepheid's system. COV2-kit revealed the Ct scores of the same gene as 27-35.5. As it was indicated before, CDC recommends the upper limit of the Ct as 37. Accordingly, all tested samples were found to be below this limit by using the COV2-kit approach. In addition, the performance of COV-kit's RdRP gene versus Cepheid's N2 gene was compared. Accordingly, the Ct value of the RdRP gene (COV2-kit) was found to be lower than N2 gene (Cepheid's), that points out the sensitivity of COV2-kit than Cepheid's system. Nevertheless, the Ct score of three COVID19 patients of N2 gene and one of RdRP gene were out of the threshold (FIG. 3b). Additionally, multiplex (triplex) rRT-PCR standardization test showed that this assay is efficient to detect three genes in the same reaction tube.

The analysis have been done in this study for RdRP and E genes as shown in simplex, duplex, and triplex gene amplifications (FIGS. 1A-1D). The results show specific targeting which means the functionality of the assay. Molecular detection of COVID-19 basically depends on the detection of RNA of the virus. Reverse transcription polymerase chain reaction (RT-PCR) is a sensitive assay for the detection of specified gene sequences encoding the proteins of the virus, such as RNA-dependent RNA polymerase (RdRP), nucleocapsid (N), envelope (E), and spike (S).

As disclosed herein, the inventors sought to bridge many challenges and weakness resulted in former assays and take benefits from their results and applications to improve novel assay.

Simplex, duplex and triplex analysis was considered to be a way to target and solve some challenges faced in previous assays as the triplex analysis provides more accuracy and avoid negative false results and possible mutation of one of viral gene. This assay is flexible for detecting potential allelic variants of the target genes. If such a variant exists, the primer/probe sequences can still identify the target genes because each primer comprises 20 nucleotides and a point mutation variant would only affect one nucleotide. Moreover, sing the assay simultaneously amplifies two viral genes, it maximizes the possibility of viral detection. Thus, in a case of an unexpected mutation that interrupts probe or primer binding, the multiplex assay can still detect at least one of the viral genes. It is also possible to modify the primer/probe sequences in the reaction mixture taking into account one or more variants of a target gene.

### Detection of RP, RdRP and N2 Genes

Corona Virus Disease 2019 (COVID-19) is a disease caused by SARS-CoV-2 that brings life to a standstill and threatens human life. Many methods are known to date to detect the virus. The real-time reverse transcription polymerase chain reaction (rRT-PCR) is one of them accepted as gold clinical standards. However, possible false-negative and false-positive results produce misleading consequences in terms of the patient's condition. Therefore, establishing sensitive primers and PCR conditions are extremely important to detect SARS-CoV-2 early and to control the spread of the disease.

In this embodiment, a novel multiplex qRT-PCR assay was designed which can detect two viral genes (N2 and RdRP) and a human gene (RP) simultaneously. Trials have been performed by using synthetic pseudoviral RNA and human target mRNA sequences as template. Also, the assay was validated by using 28 clinical SARS-COV-2 positive samples.

The performance and the accuracy of the assay was compared with the commercial kits (GeneFinder™ COVID-19 Plus RealAmp Kit (GeneFinder, Korea) and RealStar SARS-CoV-2 RT-PCR Kit 1.0 (Altona, Germany)). 28 swab specimens exhibited 100% positive percent agreement with those commercial assays. In addition, the experimental design is free from self or hetero-dimer formations that reduce sensitivity. The current multiplex rRT-PCR design provides the amplification of two viral regions in the same PCR reaction. Therefore, an accurate SARS-CoV-2 diagnostic assay was provided, which allows testing of 91 samples in 96-well plates in per run. Thanks to this strategy, fast, reliable, and easy-to-use rRT-PCR method is obtained to detect SARS-CoV-2.

Alignment of SARS-nCoV-19 genome sequences. Genomic sequences of all SARS-nCoV-19 types that have been sequenced worldwide were downloaded from the database of GISAID (Global Initiative on Sharing All Influenza Data, hypertext transfer protocol secure://worldwide web.gis.org (incorporated by reference). The comparative analyses by aligning the sequences at a base level were made with bioinformatics programs such as “Blast”, “Muscle”, and “ClustalW2”. More than 100 annotated genomes, whose genome sequence information have been determined and which include samples from Europe and Asia, were selected. In this way, virus gene targets are adjusted as sensitive, specific, and accurate as possible.

Multiplex primer/probe design. Multiplex PCR compatible primer and probe arrays that are specific to viral and human gene targets were designed using programs such as “Primer Pooler”, “PrimerPlex”, and “Primer3”. In the selection of SARS-COV-2 primers, attention was paid to the selection of genome regions that differ from other SARS-COV viruses. Therefore, primers are specific to this virus only, and exempt from possible cross reactions with other virus strains. Fluorescein amidites (FAM) labeled probe for the viral RdRP gene, hexachloro-fluorescein (HEX) labeled probe for the viral N2 gene, and a carboxyrhodamine (ROX) stained probe for the human RP gene were designed and synthesized.

Sample collection and RNA isolation. In rRT-PCR, RNA extraction is a vital pre-analytical process, which is mainly carried out using RNA extraction kits. Viral RNA was extracted from nasopharyngeal swabs in virus transport medium (VTM) which were sent to the microbiology laboratory at King Fahd Hospital of the University (KFHU), Al Khobar for SARS-CoV-2 detection. RNA extraction was performed from 280 μL of the VTM using the QIAamp Viral RNA Mini kit (Qiagen, Hilden, Germany) according to the manufacturer's instructions.

RT-qPCR reaction. The reaction mixture (20 μL) includes the following reagents: 2 of 10× Buffer, 0.25 μL of dNTPs (10 mM each), 0.2 μL of uracil-DNA glycosylase (UDG) (1 U/μL), 0.4 μL of VitaTaq® HS polymerase (2 U/μL), 0.05 μL VitaScript® Enzyme mix including M-MLV (Procomcure, Austria), 0.05 μL of Triton™ X-100 (molecular biology grade, Merck), the primer and probe mixture, and RNase/DNase-free ddH₂O up to 20 μL. The mixture for the primer and probe is varied according to the kit design. For the multiplex kit that simultaneously targeting the three genes, the final concentration of the primers/probes were adjusted as follow:

1) 10 pM for RdRP-F, 13 pM for RdRP-R, and 4 pM for RdRP-P

2) 4 pM for N2-F, 4 pM for N2-R, and 2 pM for N2-P

3) 10 pM for RP-F, 3.75 pM for RP-R, and 4 pM for RP-P

| | | SEQ |
|---|---|---|
| Primer/ | Sequence | ID |
| probe | (5-3) | NO: |
| RdRP-F | CCTCACTTGTTCTTGCTCGC | 7 |
| RdRP-R | GCCGTGACAGCTTGACAAAT | 8 |
| RdRP- | FAM-GTGAAATGGTCATGTGTGGC- | 9 |
| Probe | BHQ1 | |
| N2-F | TGAAACTCAAGCCTTACCGC | 13 |
| N-R | TATAGCCCATCTGCCTTGTG | 14 |
| N2- | HEX-ATCCATGAGCAGTGCTGAC- | 15 |
| Probe | BHQ | |
| RP-F | AGATTTGGACCTGCGAGCG | 1 |
| RP-R | GATAGCAACAACTGAATAGCCAAGGT | 2 |
| RP- | ROX-TTCTGACCTGAAGGCTCTGCGCG- | 3 |
| Probe | BHQ2 | |

The mixture was dispensed in 96-well plates (MicroAmp™ Fast Optical 96-well reaction Plate 0.1 mL, Applied Biosystems) and sealed with optical film (MicroAmp™ Optical Adhesive Film, Applied Biosystems). Pseudoviral RNAs including viral RdRP and N2 gene and human RNaseP (RP) mRNA sequences were used as the positive template. Meanwhile, RNase/DNase-free ddH₂O was added to the negative control tubes to check any contamination or primer dimer.

Quantitation experiments were performed in a real-time PCR instrument (Applied Biosystems™, 7500 Fast Real-Time PCR System). Before the operation, the instrument was calibrated by using Applied Biosystems™ 7500 Fast Real-Time PCR Systems Spectral Calibration Kit. Then, the qPCR reaction conditions were adjusted as follow: 1) Reverse transcription at 45° C. for 5 min, 2) Pre-denaturation at 95° C. for 30 sec, 3) 40 cycles of denaturation at 95° C. for 5 sec and amplification at 60° C. for 30 sec. The reporter dye channel sets as FAM for viral RdRP gene; and VIC for N gene; and ROX for human RNAseP (RP) gene. For the Applied Biosystems™ real-time PCR instrument (7500 and StepOne models), set to “passive reference” dye as “none”.

Amplification efficiency. To find out the amplification efficiency (E) of the genes, a standard curve from the dilution series of templates was prepared. Ct values versus the logarithmic amount of the template were plotted. The amplification efficiency was obtained by using the following equation:

E=100×(10^{-1/slope})

Validation of the assay. To validate the assay, we performed qRT-PCR by using commercially available SARS-CoV-2 detection kits. For this purpose, the same RNA samples (n=28) that are extracted from the COVID19 patients were used as template. The reactions were run using either GeneFinder™ COVID-19 Plus RealAmp Kit (GeneFinder, Korea) or RealStar SARS-CoV-2 RT-PCR Kit 1.0 (Altona, Germany).

Data analysis. The results were evaluated by determining the amplification curve of the target gene and the internal control gene. For the ABI 7500 device, the cycle threshold (Ct or Cq) line was automatically adjusted to ensure that the curves are all straight position. For this purpose, ABI 7500 software (v2.3) was used. The cycle threshold number≤38 with a sigmoidal curve is accepted as ‘positive’.

Standardization of the multiplex rRT-PCR. A multiplex qRT-PCR assay was developed for sensitive and accurate diagnosis of SARS-CoV-2. The assay simultaneously targets two viral genes (RdRP and N2) and one human gene (RP) as an internal control.

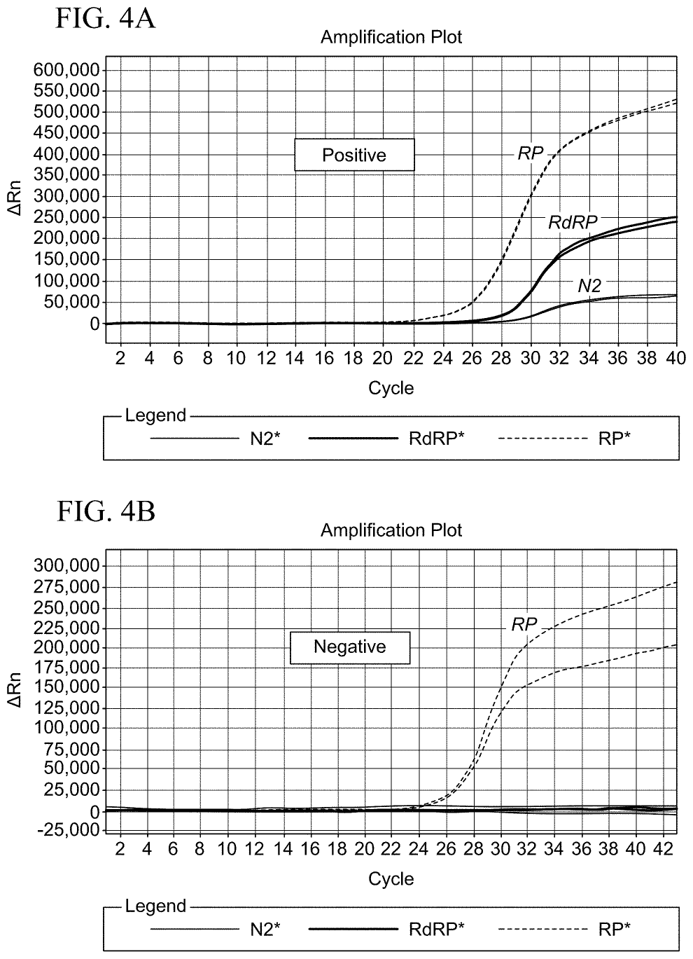

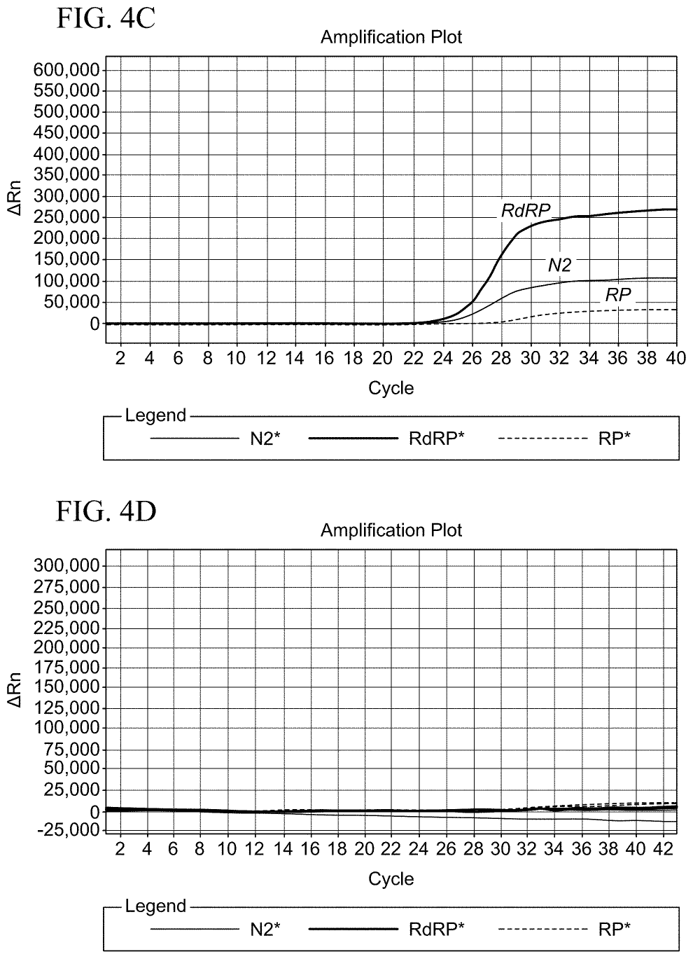

The assay tested in 28 RNA samples collected from COVID-19 positive individuals. FIGS. 4A-4D exhibit the qRT-PCR data belongs to COVID-19 positive or negative individuals.

In COVID-19 positive specimens, simultaneous amplification of RP, RdRP and N2 genes were obvious (FIG. 4A).

In the COVID-19 negative specimen, the internal control gene (RP) was the only gene amplified with a sigmoidal amplification curve (FIG. 4B).

In the positive control reactions, pseudoviral RNA including N2 and RdRP genes and a human RP gene mRNA was used. The amplification curves were obtained for all targeted genes (FIG. 4C).

In the negative control reactions, ddH₂O was used as template which led no reaction curve without primer dimer.

These results show that the multiplex primer and probe design can successfully amplify all targeted genes both in SARS-COV-2 positive specimen and synthetic positive control samples without forming primer dimers or self-amplification.

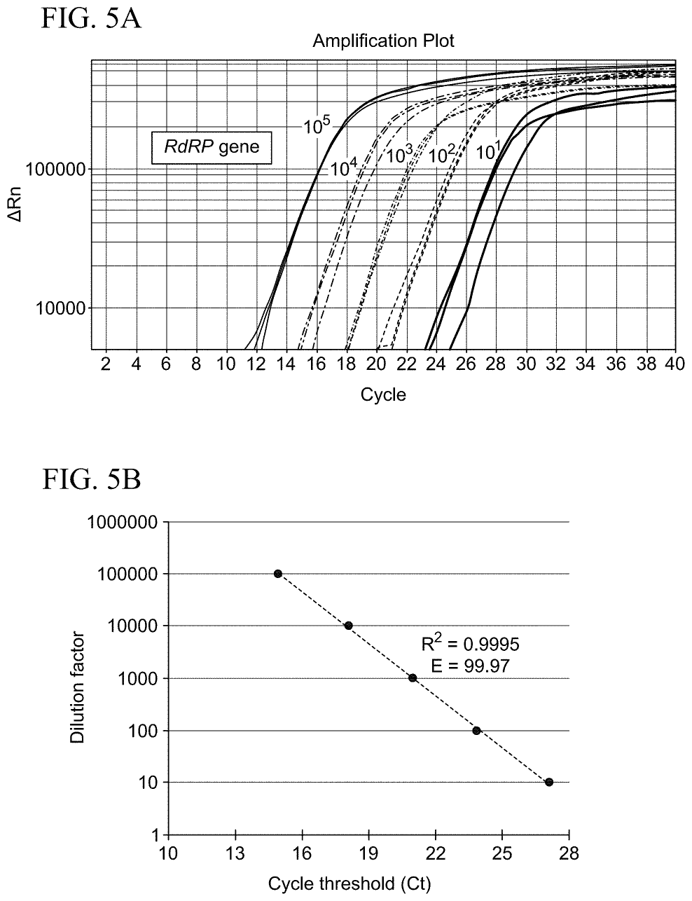

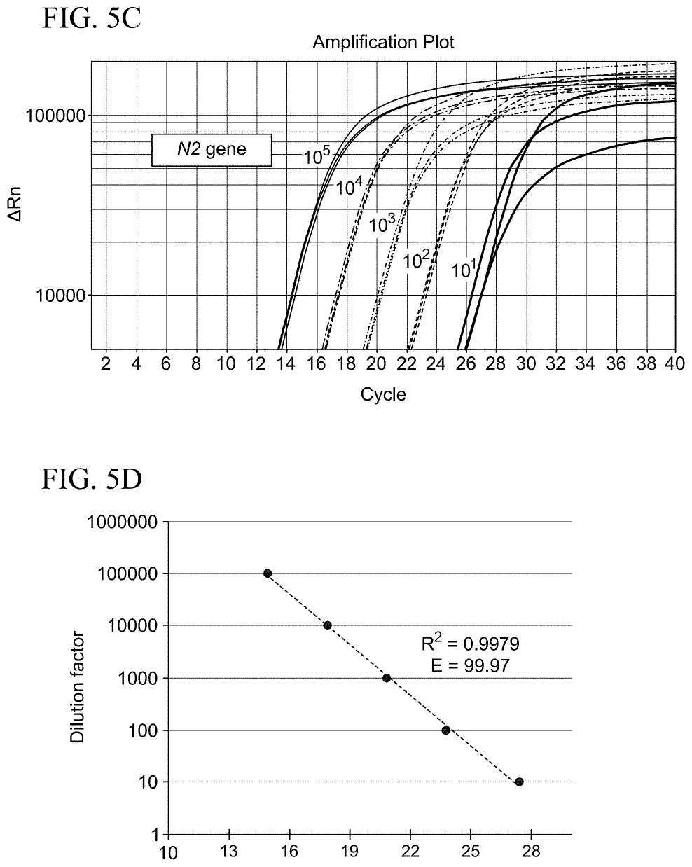

The standard curve analysis was performed to test the accuracy of the assay. For this purpose, dilution series of a clinical RNA was prepared with a dilution factor range of 10⁵ to 10¹ (FIGS. 5A-5D).

Multiplex triplicate analysis revealed that the results are consistent across technical replicates. The assay works well in all dilution ranges even if the template was diluted 10⁵ times.

The rRT-PCR efficiency for both RdRP and N2 genes was 99.97. R² value is >0.997 which shows the consistency and reliability of the assay.

Validation of the assay. The validation of the results has been carried out by using two different commercially available kits (GeneFinder™ COVID-19 Plus RealAmp Kit (GeneFinder, Korea) and RealStar SARS-CoV-2 RT-PCR Kit 1.0 (Altona, Germany)) that target different genes such as RdRP, N2, S, and E genes.

Among 28 clinically confirmed SARS-COV-2 positive samples, the current assay found 25 positives and three negatives.

The CT score of those negative samples was higher than >37 which is out of the CDC recommendations. Accordingly, the CT value≤37 is accepted as positive.

Therefore, the samples having a ≥37.01 CT score are accepted as SARS-COV-2 negative. In this case, the assay exhibited 100% positive percent agreement with those commercial assays.

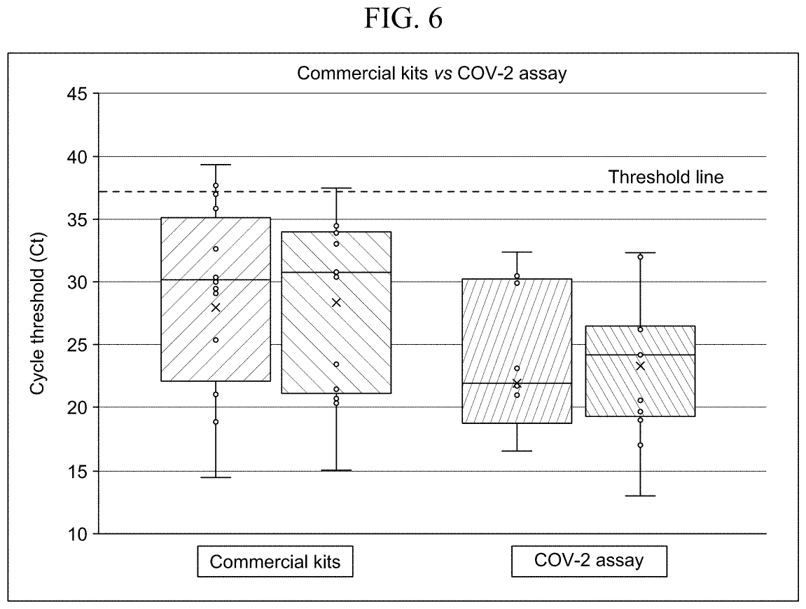

The distribution of Ct value obtained from both commercial methods and COV-2 assay are displayed in FIG. 6. Since these kits target different genes, the Ct scores of those target genes were combined. Accordingly, it can be seen that the average Ct value of the current assay is lower than those genes in representative commercial kits. This result suggests the sensitivity of the current assay which detects the genes earlier than the other tested protocols.

Limit-of-detection (LOD) and rRT-PCR efficiency. A serial dilution of synthetic RNA (5×10⁴, 5×10³, 5×10², 5×10¹, and 5×10⁰ copies/μL) was prepared to find the limit-of-detection (LOD) for RdRP and N2 genes.

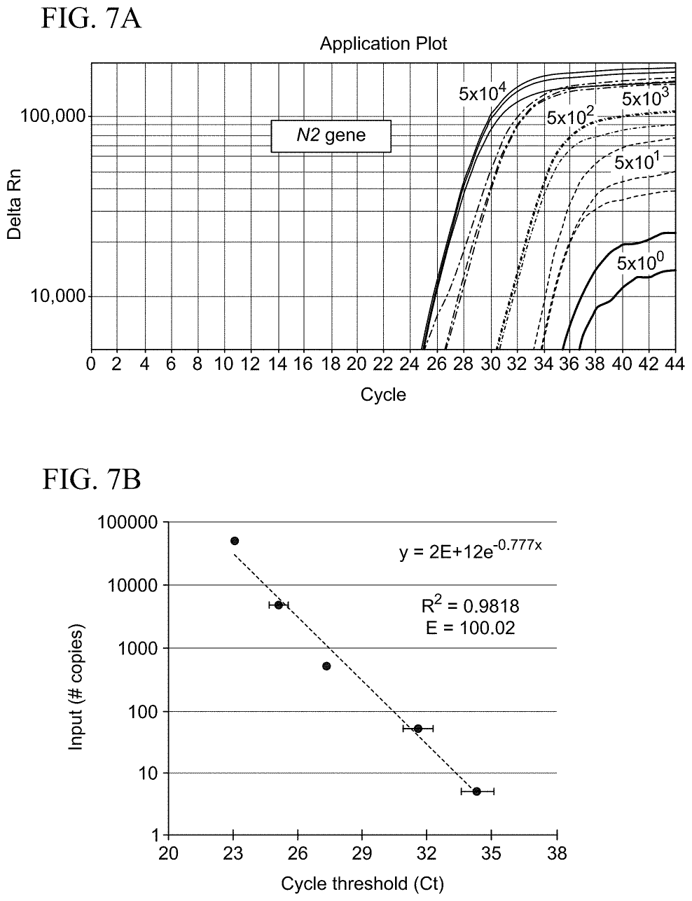

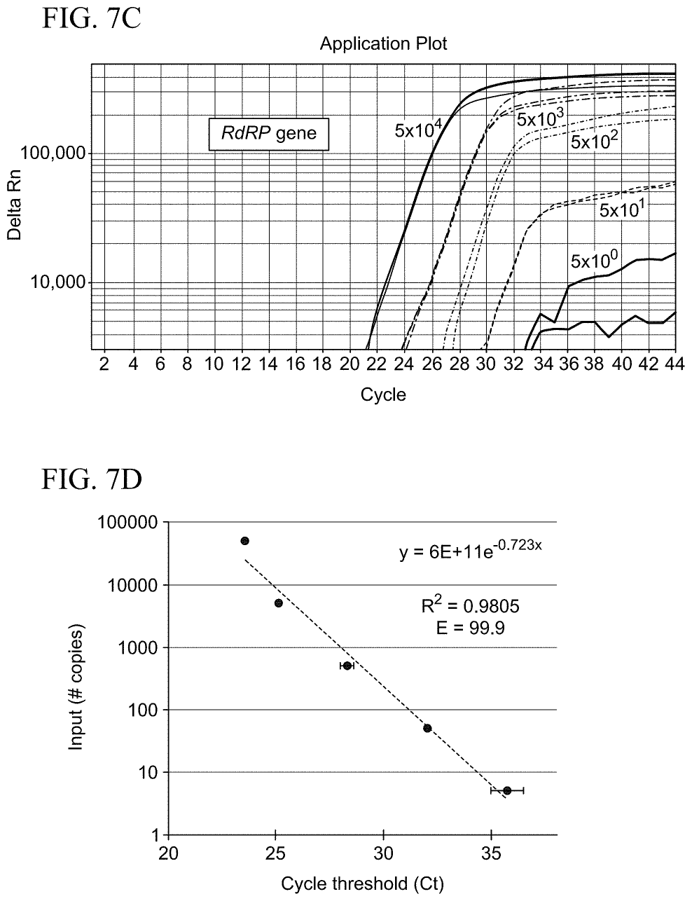

The amplification plots, the amplification efficiencies (E), and R² score are represented in FIGS. 7A-7D.

The LOD of both N2 and RdRP genes were ≤1.25 copy/μL or 5 copy/reaction. The standard curve analysis revealed that the E value of N2 and RdRP genes are 100.2 and 99.9, respectively.

The R² values are 0.9818 for the N2 and 0.9805 for the RdRP gene.

With the emergence of the COVID-19 outbreak, many methods have been developed for the diagnosis of SARS-CoV-2. In view of the many limitations of serological tests for SARS-CoV-2, the rRT-PCR method is considered the gold standard in the diagnosis of SARS-CoV-2. WHO and CDC recommend it as the diagnostic test for asymptomatic and mildly symptomatic patients. However, rRT-PCR methods also have some drawbacks such as false-negative or false-positive results and high cost. In order to eliminate or minimize those drawbacks, multiplex rRT-PCR methods have been developed that target more than one gene at the same time. However, current multiplex methods lack sensitivity.

Due to the spread of COVID-19 all over the world, there is an urgent need to develop more reliable and sensitive methods and to improve existing methods. Establishing sensitive primers and PCR conditions are extremely important to detect SARS-CoV-2 early and to control the spread of the disease. The work disclosed herein describes a multiplex rRT-PCR assay that simultaneously targets two viral (RdRP and N2) and one human internal control gene (RP). In addition, the experimental design is free from background (self- or hetero-dimer formations) and has a high sensitivity. This method provides a fast, reliable, and easy-to-use rRT-PCR method for detection of SARS-CoV-2. The rRT-PCR method disclosed herein has a higher sensitivity than currently recommended and well known assays. The validation of the assay was tested in 28 SARS-CoV-2 positive samples and showed that three samples out of 28 did that disclosed herein, it was estimated that the Ct score of the negative samples was higher than >37.01 which identified as negative. Accordingly, the Ct value equals and lower than 37 is accepted as positive. As shown above specifically targeting RdRP and N2 genes make increases the sensitivity of detecting SARS-CoV-2 in a sample.

Terminology. Terminology used herein is for the purpose of describing particular embodiments only and is not intended to be limiting of the invention.

As used herein, the singular forms “a”, “an” and “the” are intended to include the plural forms as well, unless the context clearly indicates otherwise.

As used herein, the term “and/or” includes any and all combinations of one or more of the associated listed items and may be abbreviated as “/”.

As used herein in the specification and claims, including as used in the examples and unless otherwise expressly specified, all numbers may be read as if prefaced by the word “substantially”, “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), +/−15% of the stated value (or range of values), +/−20% of the stated value (or range of values), etc. Any numerical range recited herein is intended to include all sub-ranges subsumed therein.

Disclosure of values and ranges of values for specific parameters (such as temperatures, molecular weights, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1-10 it also describes subranges for Parameter X including 1-9, 1-8, 1-7, 2-9, 2-8, 2-7, 3-9, 3-8, 3-7, 2-8, 3-7, 4-6, or 7-10, 8-10 or 9-10 as mere examples. A range encompasses its endpoints as well as values inside of an endpoint, for example, the range 0-5 includes 0, >0, 1, 2, 3, 4, <5 and 5.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

The description and specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific examples are provided for illustrative purposes of how to make and use the compositions and methods of this technology and, unless explicitly stated otherwise, are not intended to be a representation that given embodiments of this technology have, or have not, been made or tested.

All publications and patent applications mentioned in this specification are herein incorporated by reference in their entirety to the same extent as if each individual publication or patent application was specifically and individually indicated to be incorporated by reference, especially referenced is disclosure appearing in the same sentence, paragraph, page or section of the specification in which the incorporation by reference appears.

The citation of references herein does not constitute an admission that those references are prior art or have any relevance to the patentability of the technology disclosed herein. Any discussion of the content of references cited is intended merely to provide a general summary of assertions made by the authors of the references, and does not constitute an admission as to the accuracy of the content of such references.

1. A multiplex real-time reverse transcription polymerase chain reaction (rRT-PCR) method for detecting SARS-CoV-2 virus in a sample comprising:
contacting cDNA produced from SARS-CoV-2 RNA with primers that amplify human RP, viral RdRP, and viral E or N2 genes, dNTPs, and a DNA polymerase under conditions suitable for amplification of the cDNA,
contacting the amplified cDNA with fluorescent detection probes that bind to amplified human RP, viral RdRP, and viral E or N2 genes, and
measuring fluorescence as an indicator of amounts of amplified cDNA, wherein a Ct value≤37 with a sigmoidal amplification curve indicates presence of SARS-CoV-2 RNA in the sample.

2. The method of claim 1, wherein said cDNA is produced by isolating RNA from a sample and reverse transcribing SARS-CoV-2 RNA.

3. The method of claim 1, wherein said cDNA is produced by reverse transcribing purified or isolated SARS-CoV-2 RNA using an M-MLV reverse transcriptase, which is reactive at 42° C., which has RNAse H activity, but which has no detectable 3′ to 5′ exonuclease activity.

4. The method of claim 1 wherein the DNA polymerase is a Taq DNA polymerase that has a fidelity of 1× Taq, that has a standard 1 min/kb reaction speed, that exhibits a 3′-A product overhang, that has 5′ to 3′ exonuclease activity, that has undetectable 3′ to 5′ proofreading activity, and that has undetectable endonuclease activity.

5. The method of claim 1, wherein the PCR reaction mixture comprises Triton-X 100 or dimethyl sulfoxide (DMSO), and Uracil-DNA glycosylase (UDG).

6. The method of claim 1, wherein said primers that amplify human RP, viral RdRP, and viral E genes comprise:
RP forward primer
(SEQ ID NO: 1)
AGATTTGGACCTGCGAGCG
and
RP reverse primer
(SEQ ID NO: 2)
GATAGCAACAACTGAATAGCCAAGGT;
RdRP forward primer
(SEQ ID NO: 4)
GTCATGTGTGGCGGTTCACT
and
RdRP reverse primer
(SEQ ID NO: 5)
CAACACTATTAGCATAAGCAGTTGT;
or
RdRP forward primer
(SEQ ID NO: 7)
CCTCACTTGTTCTTGCTCGC
and
reverse primer
(SEQ ID NO: 8)
GCCGTGACAGCTTGACAAAT;
and
E forward primer
(SEQ ID NO: 10)
GGAAGAGACAGGTACGTTAATA
and
E reverse primer
(SEQ ID NO: 11)
AGCAGTACGCACACAATCGAA.

7. The method of claim 1, wherein said primers that amplify human RP, viral RdRP, and viral N2 genes comprise:
RP forward primer
(SEQ ID NO: 1)
AGATTTGGACCTGCGAGCG
and
RP reverse primer
(SEQ ID NO: 2)
GATAGCAACAACTGAATAGCCAAGGT;
RdRP forward primer
(SEQ ID NO: 4)
GTCATGTGTGGCGGTTCACT
and
RdRP reverse primer
(SEQ ID NO: 5)
CAACACTATTAGCATAAGCAGTTGT;
or
RdRP forward primer
(SEQ ID NO: 7)
CCTCACTTGTTCTTGCTCGC
and
reverse primer
(SEQ ID NO: 8)
GCCGTGACAGCTTGACAAAT;
and
N2 forward primer
(SEQ ID NO: 13)
GAAACTCAAGCCTTACCGC
and
N2 reverse primer
(SEQ ID NO: 14)
TATAGCCCATCTGCCTTGTG.

8. The method of claim 1, wherein the fluorescent detection probes are each labeled with a different fluorescent moiety and comprise:
for RP:
(SEQ ID NO: 3)
TTCTGACCTGAAGGCTCTGCGCG,
for RdRP:
(SEQ ID NO: 6)
CAGGTGGAACCTCATCAGGAGATGC
or
(SEQ ID NO: 9)
TTCTGACCTGAAGGCTCTGCGCG;
and
for E:
(SEQ ID NO: 12)
ACACTAGCCATCCTTACTGCGCTTCG.

9. The method of claim 1, wherein the fluorescent detection probes are each labeled with a different fluorescent moiety and consist of:
for RP:
(SEQ ID NO: 3)
ROX-TTCTGACCTGAAGGCTCTGCGCG-BHQ2,
for RdRP:
(SEQ ID NO: 6)
FAM-CAGGTGGAACCTCATCAGGAGATGC-BHQ1
or
(SEQ ID NO: 9)
FAM-TTCTGACCTGAAGGCTCTGCGCG-BHQ1-;
and
for E:
(SEQ ID NO: 12)
REX-ACACTAGCCATCCTTACTGCGCTTCG-BHQ1.

10. The method of claim 1, wherein the fluorescent detection probes are each labeled with a different fluorescent moiety and comprise:
for RP:
(SEQ ID NO: 3)
TTCTGACCTGAAGGCTCTGCGCG,
for RdRP:
(SEQ ID NO: 6)
CAGGTGGAACCTCATCAGGAGATGC
or
(SEQ ID NO: 9)
TTCTGACCTGAAGGCTCTGCGCG;
and
for N2:
(SEQ ID NO: 15)
ATCCATGAGCAGTGCTGAC.

11. The method of claim 1, wherein the fluorescent detection probes are each labeled with a different fluorescent moiety and consist of:
for RP:
(SEQ ID NO: 3)
ROX-TTCTGACCTGAAGGCTCTGCGCG-BHQ2,
for RdRP:
(SEQ ID NO: 6)
FAM-CAGGTGGAACCTCATCAGGAGATGC-BHQ1
or
(SEQ ID NO: 9)
FAM-TTCTGACCTGAAGGCTCTGCGCG-BHQ1;
and
for N2:
(SEQ ID NO: 15)
HEX-ATCCATGAGCAGTGCTGAC-BHQ1.

12. The method of claim 1 that has a running time of 45 minutes or less.

13. The method of claim 1 that has a limit of detection (LOD) for the RdRP gene of ≤10 copy/μL.

14. The method of claim 1 that has a limit of detection (LOD) for the E gene of ≤10 copy/μL.

15. The method of claim 1 that has a limit of detection (LOD) for the N2 gene of ≤10 copy/μL.

16. The method of claim 1 that has an R² of at least 0.98 for the E gene and an R2 of at least 0.97 for the RdRP gene and that has an efficiency (E) of at least 0.99 for each of the E and RdRP genes.

17. The method of claim 1 which has an R² of at least 0.98 for the N2 gene and an R2 of at least 0.97 for the RdRP gene and that has an efficiency (E) of at least 0.99 for each of the N2 and RdRP genes.

18. A kit comprising reverse transcriptase, DNA polymerase, dNTPs a medium suitable for reverse transcription of SARS-CoV-2 RNA into cDNA, a medium suitable for amplification of cDNA, primers suitable for amplification of human RP and SARS-CoV-2 viral RdRP, and viral E genes, wherein said primers comprise:
RP forward primer AGATTTGGACCTGCGAGCG (SEQ ID NO: 1) and RP reverse primer GATAGCAACAACTGAATAGCCAAGGT (SEQ ID NO: 2);
RdRP forward primer GTCATGTGTGGCGGTTCACT (SEQ ID NO: 4) and RdRP reverse primer CAACACTATTAGCATAAGCAGTTGT (SEQ ID NO: 5); or RdRP forward primer CCTCACTTGTTCTTGCTCGC (SEQ ID NO: 7) and reverse primer GCCGTGACAGCTTGACAAAT (SEQ ID NO: 8); and
E forward primer GGAAGAGACAGGTACGTTAATA (SEQ ID NO: 10) and E reverse primer AGCAGTACGCACACAATCGAA (SEQ ID NO: 11); at least one container, and, optionally, a thermocycler and/or a fluorescence detector; and/or
primers suitable for amplification of human RP and SARS-CoV-2 viral RdRP, and viral E genes, wherein said primers comprise:
RP forward primer AGATTTGGACCTGCGAGCG (SEQ ID NO: 1) and RP reverse primer GATAGCAACAACTGAATAGCCAAGGT (SEQ ID NO: 2);
RdRP forward primer GTCATGTGTGGCGGTTCACT (SEQ ID NO: 4) and RdRP reverse primer CAACACTATTAGCATAAGCAGTTGT (SEQ ID NO: 5); or RdRP forward primer CCTCACTTGTTCTTGCTCGC (SEQ ID NO: 7) and reverse primer GCCGTGACAGCTTGACAAAT (SEQ ID NO: 8); and
N2 forward primer GAAACTCAAGCCTTACCGC (SEQ ID NO: 13) and N2 reverse primer TATAGCCCATCTGCCTTGTG (SEQ ID NO: 14); and, optionally,
at least one container, a thermocycler, a fluorescence detector, and/or instructions for use in detecting SARS-CoV-2.

19. The method of claim 18, further comprising
fluorescent detection probes which are each labeled with a different fluorescent moiety and which comprise:
for RP: TTCTGACCTGAAGGCTCTGCGCG (SEQ ID NO:3),
for RdRP: CAGGTGGAACCTCATCAGGAGATGC (SEQ ID NO: 6) or GTGAAATGGTCATGTGTGGC (SEQ ID NO: 9); and
for E: ACACTAGCCATCCTTACTGCGCTTCG (SEQ ID NO: 12); and/or
fluorescent detection probes which are each labeled with a different fluorescent moiety and which comprise:
for RP:
(SEQ ID NO: 3)
TTCTGACCTGAAGGCTCTGCGCG,
for RdRP:
(SEQ ID NO: 6)
CAGGTGGAACCTCATCAGGAGATGC
or
(SEQ ID NO: 9)
GTGAAATGGTCATGTGTGGC;
and
for N2:
(SEQ ID NO: 15)
ATCCATGAGCAGTGCTGAC.

20. A method for preventing or treating an infection by SARS-CoV-2 comprising selecting a subject in need of vaccination or treatment for SARS-CoV-2 by detecting SARS-CoV-2 RNA in a biological sample from the subject according to the method of claim 1, and vaccinating or treating the subject for SARS-CoV-2 when SARS-CoV-2 RNA is detected or vaccinating or prophylactically treating the subject when SARS-CoV-2 RNA is not detected.


SEQUENCE LISTING (15 sequences)

SEQ ID NO: 1 - DNA, 19 bp, Homo sapiens
  misc_feature (1)..(10): RP forward primer
agatttggacctgcgagcg

SEQ ID NO: 2 - DNA, 26 bp, Homo sapiens
  misc_feature (1)..(26): RP reverse primer
gatagcaacaactgaatagccaaggt

SEQ ID NO: 3 - DNA, 23 bp, Homo sapiens
  misc_feature (1)..(23): RP probe
ttctgacctgaaggctctgcgcg

SEQ ID NO: 4 - DNA, 20 bp, Artificial Sequence
  RdRP forward primer 1
gtcatgtgtggcggttcact

SEQ ID NO: 5 - DNA, 25 bp, Artificial Sequence
  RdRP reverse primer 1
caacactattagcataagcagttgt

SEQ ID NO: 6 - DNA, 25 bp, Artificial Sequence
  RdRP probe 1
caggtggaacctcatcaggagatgc

SEQ ID NO: 7 - DNA, 20 bp, Artificial Sequence
  RdRP forward primer 2
cctcacttgttcttgctcgc

SEQ ID NO: 8 - DNA, 20 bp, Artificial Sequence
  RdRP reverse primer 2
gccgtgacagcttgacaaat

SEQ ID NO: 9 - DNA, 23 bp, Artificial Sequence
  RdRP probe 2
ttctgacctgaaggctctgcgcg

SEQ ID NO: 10 - DNA, 22 bp, Artificial Sequence
  E forward primer
ggaagagacaggtacgttaata

SEQ ID NO: 11 - DNA, 21 bp, Artificial Sequence
  E reverse primer
agcagtacgcacacaatcgaa

SEQ ID NO: 12 - DNA, 26 bp, Artificial Sequence
  E probe
acactagccatccttactgcgcttcg

SEQ ID NO: 13 - DNA, 19 bp, Artificial Sequence
  N2 forward primer
gaaactcaagccttaccgc

SEQ ID NO: 14 - DNA, 20 bp, Artificial Sequence
  N2 reverse primer
tatagcccatctgccttgtg

SEQ ID NO: 15 - DNA, 19 bp, Artificial Sequence
  N2 probe
atccatgagcagtgctgac